(19) United States
(12) Patent Application Publication
(10) Pub. No.: US 2022/0202571 A1
(43) Pub. Date: Jun. 30, 2022

(54) PAPILLARY MUSCLE APPROXIMATION AND VENTRICULAR RESTORATION

(71) Applicant: YALE UNIVERSITY, New Haven, CT (US)

(72) Inventor: Pramod Bonde, Woodbridge, CT (US)

(21) Appl. No.: 17/609,241

(22) PCT Filed: May 7, 2020
(86) PCT No.: PCT/US20/31917

§ 371 Date: Nov. 5, 2021

Related U.S. Application Data
(60) Provisional application No. 62/844,737, filed on May 7, 2019.

(51) Int. Cl. A61F 2/24; A61F 2/848

(52) U.S. Cl. CPC A61F 2/2487; A61F 2/2457; A61F 2220/0016; A61F 2210/009; A61F 2/848

(57) ABSTRACT

The present invention provides ventricular restoration devices and snare devices as part of a papillary muscle approximation and ventricular restoration (PAP-VR) system and methods for using the same to repair mitral regurgitation (MR). The ventricular restoration devices include two collapsible anchors positioned at opposing ends of a collapsible stent, wherein the devices are threaded through a subject's anatomy such that the anchors rest outside of tissue and are held taut by the stent inbetween. The snare devices comprise an out-of-plane curved construction to navigate magnetized trapping lines around difficult to reach anatomy to lasso and tighten anatomical structures.

### CROSS-REFERENCE TO RELATED APPLICATIONS

This application claims priority to U.S. Provisional Patent Application No. 62/844,737, filed May 7, 2019, the contents of which are incorporated by reference herein in its entirety.

### BACKGROUND OF THE INVENTION

Mitral valve regurgitation (MR) occurs when the mitral valve of the heart leaks and results in significant morbidity and mortality. MR is a rapidly progressing disease that increases load and stress in the heart, leading to muscle damage and muscle loss, dysfunction and dilation of the left ventricle, and exacerbation of MR.

The mitral valve is composed of an annulus surrounding a bicuspid valve, with each leaflet of the valve anchored to papillary muscles in the left ventricle by chordae tendinae. Current technologies that aim to repair MR are focused on augmenting the annulus, leaflets, or chordae tendinae. Traditionally, open heart surgery is used to implant an annuloplasty ring to reinforce the annulus. In severe cases, the entire mitral valve can be replaced with an artificial valve. However, these procedures require that the heart be placed on cardiopulmonary bypass or a heart-lung machine and for the left ventricle to be opened, and sizing the annuloplasty ring for each patient is a cumbersome process. Recently, minimally invasive techniques have been developed as an alternative to annuloplasty. For example, the Abbott MitraClip is a minimally invasive device that clips the anterior and posterior mitral valve leaflets together to minimize the valve orifice. However, the MitraClip does not perform as well as an annuloplasty and only addresses one aspect of MR. In another example, the NeoChord DS1000 is an implantable artificial chordae tendinae that can be implanted without cardiopulmonary bypass. However, it is implanted transapically and still requires some trauma to the heart. While additional minimally invasive techniques and devices are under development, the focus remains on the annulus, leaflets, and chordae tendinae.

There is a need in the art for improved devices and methods for the repair of MR. The present invention addresses this need.

### SUMMARY OF THE INVENTION

In one aspect, the present invention provides a papillary muscle approximation and ventricular restoration (PAP-VR) system comprising: at least one ventricular restoration device, each ventricular restoration device comprising a collapsible anchor attached to opposing ends of a collapsible stent; and at least one snare device, each snare device comprising an elongate snare body having a lumen connecting a proximal opening and a distal opening, and a trapping line positioned within the lumen, each trapping line having a magnet attached to a distal end.

In one embodiment, the at least one ventricular restoration device further comprises one or more magnetic anchors, wherein the one or more magnetic anchors are configured to form a band or ring like shape upon magnetic attraction to each other. In one embodiment, the one or more magnetic anchors each comprise a papillary muscle-facing surface comprising a plurality of barbs or hooks. In one embodiment, the one or more magnetic anchors are linked together by a fabric, a mesh, or a sleeve.

In one embodiment, each of the anchors has a shape selected from the group consisting of: a donut shape, a bracket shape, a claw shape, a barb shape, an orthogonal rod shape, a cross shape, a multi-legged shape, a disc shape, a clover shape, and combinations thereof. In one embodiment, the at least one ventricular restoration device is constructed from a material selected from the group consisting of: nitinol, PTFE, polyester, and silicone.

In one embodiment, a distance between each opposing anchor is adjustable along a length of each stent. In one embodiment, each snare device comprises a distal hook end. In one embodiment, each snare device comprises a main axis aligned along a length of each snare body. In one embodiment, each snare body comprises at least one out-of-axis or out-of-plane angulation that deviates from the main axis. In one embodiment, the at least one out-of-axis or out-of-plane angulation is positioned along the length of the snare body, at a junction between a distal hook end, along a curvature of the distal hook end, and combinations thereof.

In another aspect, the present invention provides a method of repairing mitral regurgitation in a subject, the method comprising the steps of: providing at least one snare device, each snare device having a snare body with a lumen extending between a proximal opening and a distal opening and a trapping line positioned within each lumen, each trapping line having a magnet at a distal tip; introducing the at least one snare device into a subject's ventricle; hooking a distal end of the at least one snare device around a pair of papillary muscles; adhering the magnet of each trapping line to each other, thereby lassoing the trapping lines around the pair of papillary muscles; retracting each snare body from the trapping lines; tightening the trapping lines around the papillary muscles; approximating a distance between the papillary muscles; selecting at least one ventricular restoration device, each ventricular restoration device having a collapsible anchor attached to opposing ends of a collapsible stent, wherein each stent has a length sized to span the distance between the papillary muscles; and piercing the at least one ventricular restoration device through the papillary muscles, such that the collapsible anchors rest against opposing tissue surfaces of the papillary muscles and the stent holds the anchors and the opposing tissue surfaces taut. In one embodiment, the ventricle is accessed from a transaortic approach, a transmitral approach, or a transventricular approach.

In one embodiment, the approximating step comprises a step of calculating a total length of trapping line lassoed around the pair of papillary muscles such that the total length forms a circumference of a substantially circular shape. In one embodiment, the selection step selects at least one ventricular restoration device having a length substantially equal to a diameter of the substantially circular shape.

In one embodiment, the method further comprises a step of piercing at least one ventricular restoration device through a papillary muscle and a valve annulus. In one embodiment, the method further comprises a step of piercing at least one ventricular restoration device through a papillary muscle and a valve leaflet. In one embodiment, the method further comprises a step of piercing at least one ventricular restoration device through a papillary muscle and a heart wall, such that the papillary muscle is shifted out of its natural plane.

In one embodiment, the at least one ventricular restoration device comprises one or more magnetic anchors. In one embodiment, the method further comprises a step of implanting at least one magnetic anchor into an adjacent heart wall, such that magnetic attraction between the at least one ventricular restoration device and the at least one magnetic anchor in the heart wall biases the position of the at least one ventricular restoration device.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1 depicts exemplary ventricular restoration devices and a diagram of a ventricle.

FIG. 2 depicts a side view (left) and a top-down view (right) of an exemplary ventricular restoration device secured to a pair of papillary muscles.

FIG. 3 depicts several exemplary ventricular restoration devices.

FIG. 4 depicts side views (left column) and top-down views (right column) of several exemplary ventricular restoration devices, each secured to a pair of papillary muscles.

FIG. 5 depicts exemplary snare devices and trapping lines.

FIG. 6 depicts a magnified view of the distal end of an exemplary snare device within a catheter with a loaded trapping line.

FIG. 7 depicts a top-down view (left) of an exemplary snare device lassoing a pair of papillary muscles, an exemplary manipulator (top right) with a measurement display, and a diagram (bottom right) approximating the diameter of a lassoed section with the length of an exemplary ventricular restoration device.

FIG. 8 depicts a flowchart of an exemplary method of repairing mitral regurgitation (MR).

FIG. 9A through FIG. 9D illustrate exemplary method steps of repairing MR from a transaortic approach.

FIG. 10 depicts a top-down view of repairing MR from a transaortic approach.

FIG. 11 depicts a top-down view of repairing MR from a transmitral approach.

FIG. 12 depicts a side view of repairing MR from a transventricular approach.

FIG. 13 depicts various configurations of securing a pair of papillary muscles using ventricular restoration devices.

FIG. 14 depicts a diagram of a ventricle (left) and the use of the ventricular restoration devices to secure the mitral valve leaflets from the side (top right) and across the valve (bottom right).

FIG. 15 depicts the use of the ventricular restoration devices to secure a papillary muscle to the annulus (left), to secure a papillary muscle to a leaflet as chordae tendinae augmentation (middle), and to modify the shape of the ventricle (right).

### DETAILED DESCRIPTION

The present invention provides ventricular restoration devices and snare devices as part of a papillary muscle approximation and ventricular restoration (PAP-VR) system and methods for using the same to repair mitral regurgitation (MR). The ventricular restoration devices include two collapsible anchors positioned at opposing ends of a collapsible stent, wherein the devices are threaded through a subject's anatomy such that the anchors rest outside of tissue and are held taut by the stent in-between. The snare devices comprise an out-of-plane curved construction to navigate magnetized trapping lines around difficult to reach anatomy to lasso and tighten anatomical structures.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range.

### Ventricular Restoration Device

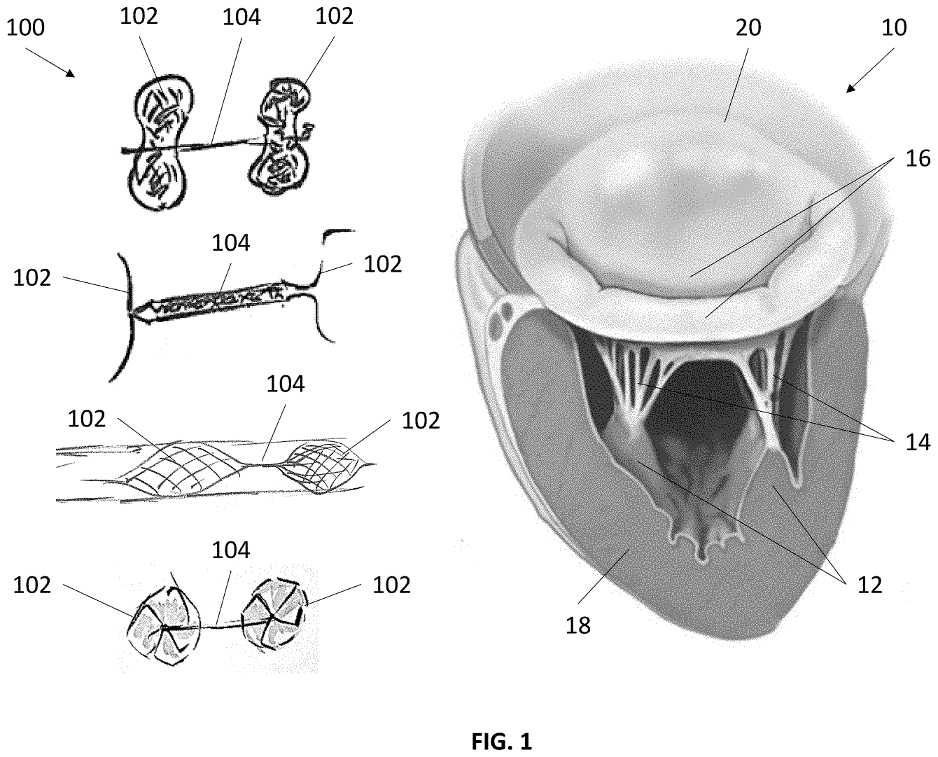

Referring now to FIG. 1, an exemplary ventricular restoration device 100 is depicted. Device 100 comprises two collapsible anchors 102 attached to opposing ends of a collapsible stent 104. Anchors 102 and stent 104 can each have a mesh, wire, or scaffold construction, such that anchors 102 and stent 104 are collapsible into a narrow configuration to facilitate insertion through a narrow lumen in minimally invasive procedures, as well as expandable at a site of implantation. In some embodiments, stent 104 can simply be a length of wire or string connecting two anchors 102. Anchors 102 and stent 104 can be constructed from any suitable material, such as Nitinol, PTFE, polyester, silicone, and the like. In certain embodiments, anchors 102, stent 104, or combinations thereof comprise a covering or coating, which can have a biological (such as pericardium or engineered tissue scaffold), artificial (such as a polymer), or a biological and artificial hybrid construction. The covering or coating can include one or more therapeutics that promote biocompatibility, healing, and the like.

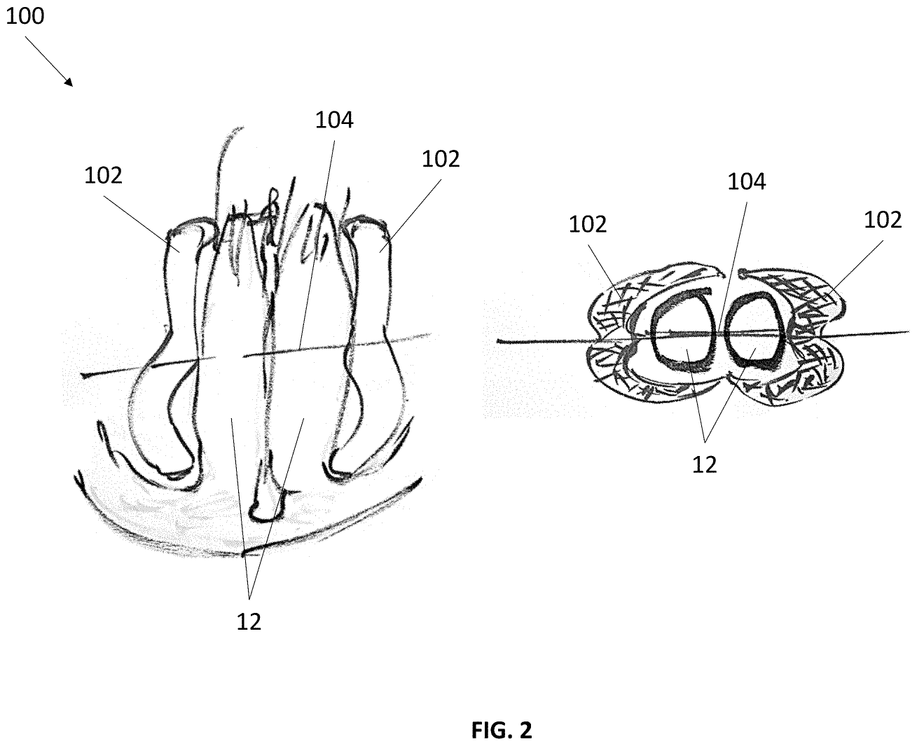

Referring now to FIG. 2, device 100 is configured to traverse the anatomy of a subject such that each anchor 102 rests against a surface of tissue and stent 104 maintains a taut connection between each anchor 102 to maintain a distance between the tissue surfaces. For example, FIG. 2 depicts device 100 securing a pair of papillary muscles 12 together. Anchors 102 rests against opposing surfaces of the papillary muscles 12, and stent 104 has a length selected to bring the papillary muscles 12 closely together.

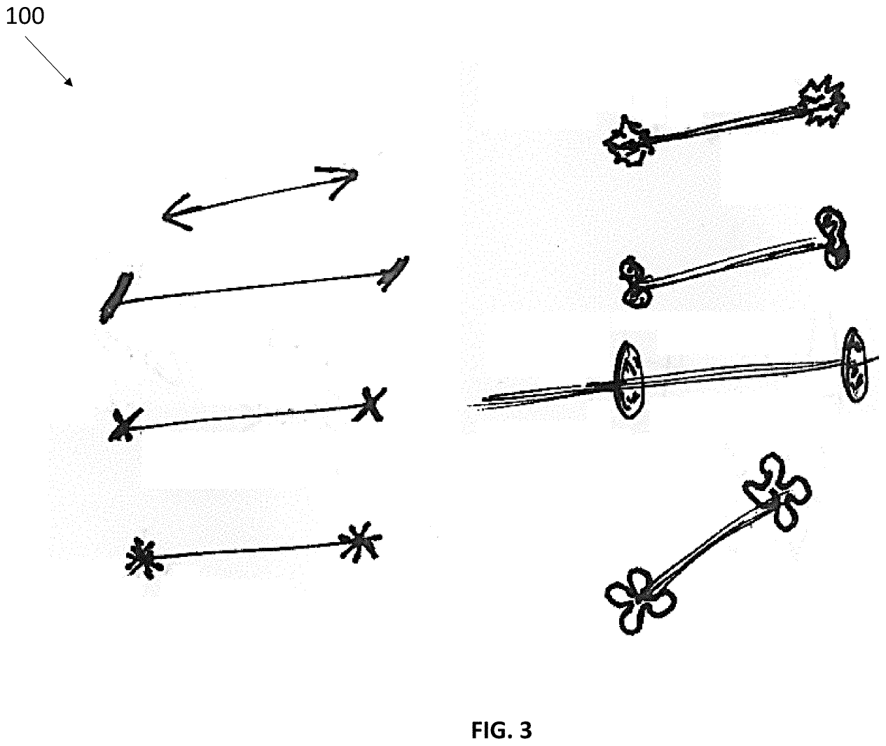

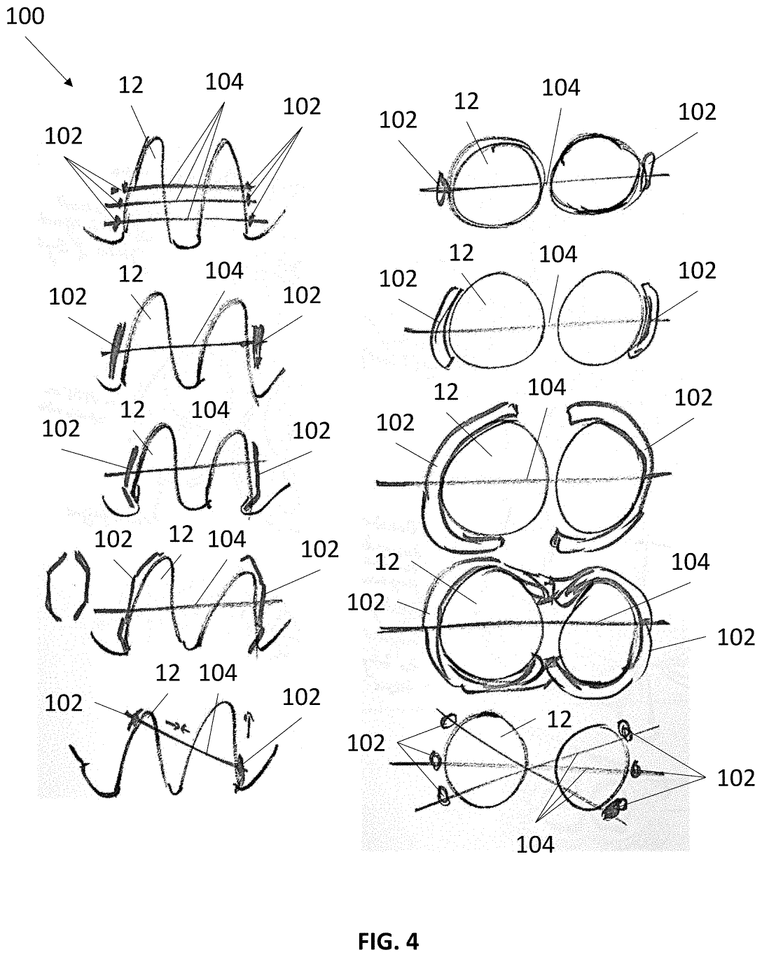

Anchors 102 can have any desired shape or configuration. For example, FIG. 1 (left) depicts four non-limiting examples of anchor 102 shapes, which from top to bottom comprise a side view of a donut shape, a side view of a claw shape, a side view of a basket shape, and a perspective view of a fan shape. The exemplary device 100 in FIG. 2 depicts a bracket shape (left) having extensions that apply pressure to the superior and inferior ends of papillary muscles 12 and a bracket shape (right) having extensions that wrap around a pair of papillary muscles 12. FIG. 3 depicts additional non-limiting examples of anchor 102 shapes. In the left column, from top to bottom: a barbed design, an orthogonal rod design; a cross design, and a multi-legged design. In the right column, from top to bottom: a ball design, a donut design, a disc design, and a clover design. In various embodiments, the distance between opposing anchors 102 is adjustable. The adjustable distance can be achieved by any of several means, such as by modifying a length of stent 104, or by modifying the position of an anchor 102 along a length of stent 104. FIG. 4 depicts non-limiting configurations of device 100 secured to papillary muscles 12. In the left column, from top to bottom: multiple devices 100 used in tandem, each device 100 having mini anchors 102; a single device 100 having large anchors 102; a single device 100 having bracket-shaped anchors 102 with inferior extensions; a single device 100 having bracket-shaped anchors 102 with inferior and superior extensions; a single device 100 securing a superior end of a first papillary muscle 12 and an inferior end of a second papillary muscle 12. In the right column, from top to bottom: a single device 100 having mini anchors 102; a single device 100 having large anchors 102; a single device 100 having bracket-shaped anchors 102 that wrap around their respective papillary muscles; a single device 100 having bracket-shaped anchors 102 that wrap completely around both papillary muscles; multiple devices 100 used in tandem, each device 100 having mini anchors 102.

In some embodiments, device 100 comprises an array of magnetic anchors 102, wherein the array of magnetic anchors 102 can be positioned around papillary muscles 12 such that magnetic attraction between each magnetic anchor 102 forms a substantially band or ring like shape that wraps around and compresses papillary muscles 12 towards each other. In some embodiments, the array of magnetic anchors 102 can be linked together, such as with a fabric, a mesh, or one or more stents 104. In some embodiments, the array of magnetic anchors 102 can be secured to a sleeve or band. In certain embodiments, the array of magnetic anchors 102 can comprise a covering or coating, which can have a biological (such as pericardium or engineered tissue scaffold), artificial (such as a polymer), or a biological and artificial hybrid construction. The covering or coating can include one or more therapeutics that promote biocompatibility, healing, and the like. In some embodiments, the array of magnetic anchors 102 can include one or more structures that enhance grip on tissue. For example, each magnetic anchor 102 in the array can comprise a papillary muscle-facing surface having a plurality of barbs or hooks, such that upon physical contact with papillary muscles 12, the plurality of barbs or hooks penetrates papillary muscles 12 and prevents the array of magnetic anchors 102 from sliding out of position.

### Snare Device

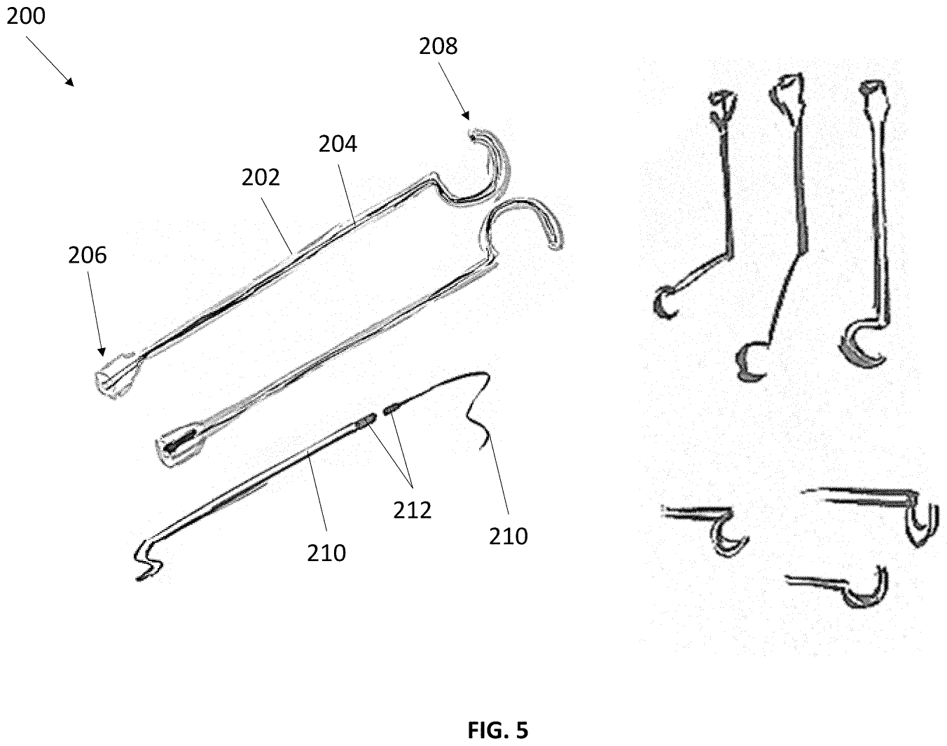

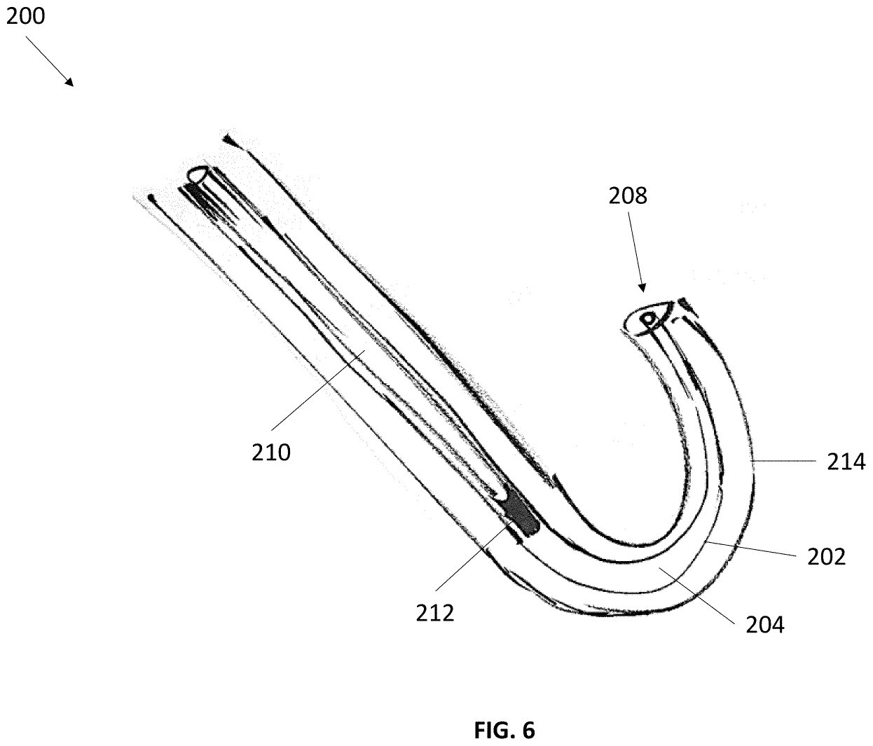

Referring now to FIG. 5 and FIG. 6, an exemplary snare device 200 is depicted. Device 200 comprises an elongate snare body 202 having a lumen 204 connecting a proximal opening 206 and a distal opening 208. Snare body 202 comprises a hook shape at its distal end suitable for navigating difficult-to-access anatomical structures, such as around papillary muscles. Snare body 202 comprises a main axis aligned along its length, and snare body 202 can comprise one or more out-of-axis and out-of-plane angulations, as depicted in FIG. 5 (right). The out-of-axis and out-of-plane angulations can occur anywhere along snare body 202, at the junction between snare body 202 and the hook shape end, along the curvature of the hook end, and combinations thereof. In various embodiments, each out-of-axis and out-of-plane angulations can deviate from an adjacent axis by about 10°, 20°, 30°, 40°, 50°, 60°, 70°, 80°, 90°, 100°, 110°, 120°, 130°, 140°, 150°, 160°, or 170°.

Device 200 further comprises trapping lines 210 having magnets 212 positioned at their distal ends, wherein trapping lines 210 are sized to fit within lumen 204, such as about a 2-4 mm wide lumen 204. Trapping lines 210 can be constructed from any substantially flexible material, such as a wire, tape, ribbon, or tube constructed from nylon, PTFE, linen, cotton, polypropylene, and the like, that is non-absorbable, non-stretchable, and capable of navigating and being manipulated through lumen 204 of snare body 202. Magnets 212 can be any suitable magnet, such as neodymium magnets, samarium cobalt magnets, ceramic magnets, or ferrite magnets.

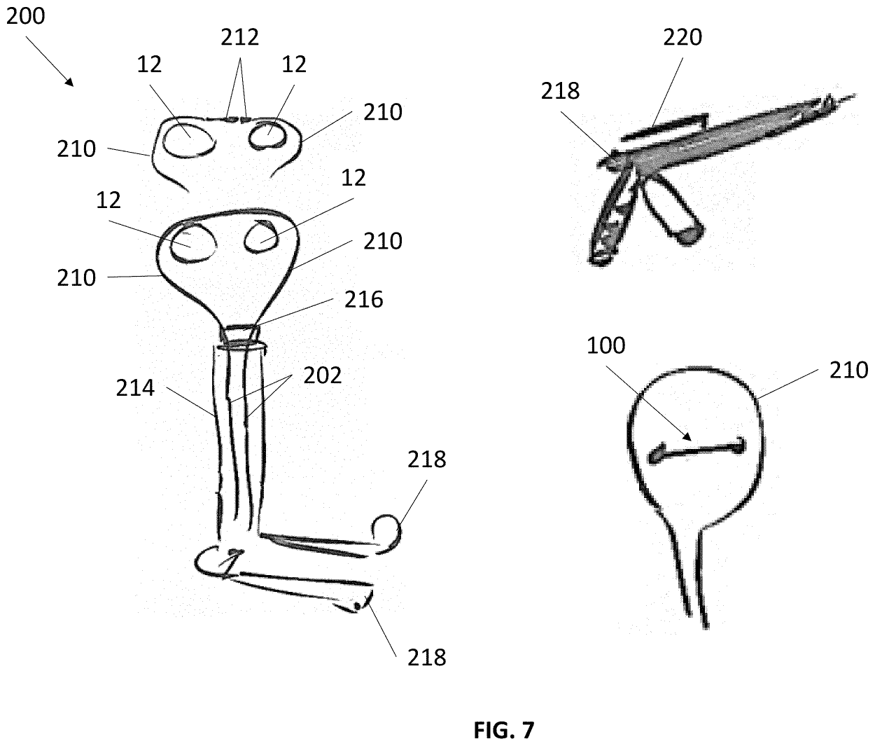

FIG. 7 depicts an exemplary a pair of devices 200 deployed through a catheter 214 for the purpose of lassoing a pair of papillary muscles 12 using trapping lines 210. The pair of devices 200 are introduced adjacent to the papillary muscles 12 and hooked around the papillary muscles 12 via manipulators 218, whereupon trapping lines 210 are pushed out of snare body 202 and their respective magnets 212 meet. The pair of devices 200 can be held together by clip 216 to improve stability. After the trapping lines 210 are secured to each other by magnets 212, each snare body 202 can be withdrawn into catheter 214 by manipulators 218 such that trapping lines 210 remain lassoed around the papillary muscles 12. Trapping lines 210 can then be retracted to remove any slack in trapping lines 210, which lassos papillary muscles 12 with a desired degree of tightness. Clip 216 can be used to improve the lasso grip of trapping lines 210 on papillary muscles 12. Given that trapping lines 210 having a known length and catheter 214 has a known length, the length of trapping lines 210 that is lassoed around the papillary muscles 12 can be calculated by subtracting the length of catheter 214 from the length of trapping lines 210 that are in catheter 214 and lassoed around the papillary muscles 12. The length of trapping lines 210 lassoed around the papillary muscles 12 can be described as a circumference of a substantially circular shape, wherein the substantially circular shape represents the size and shape of the compressed papillary muscles 12 such that a diameter of the substantially circular shape is representative of a length of a device 100 needed to span the width of the compressed papillary muscles 12. In some embodiments, the length of trapping lines 210 that is lassoed around the papillary muscles 12 can be shown on a measurement display 220 positioned on a manipulator 218. Having known the length of trapping lines 210 that is lassoed around the papillary muscles 12, a distance between opposing surfaces of the lassoed papillary muscles 12 can be approximated, which can be used to select an appropriately sized device 100.

### Method of Repairing Mitral Regurgitation

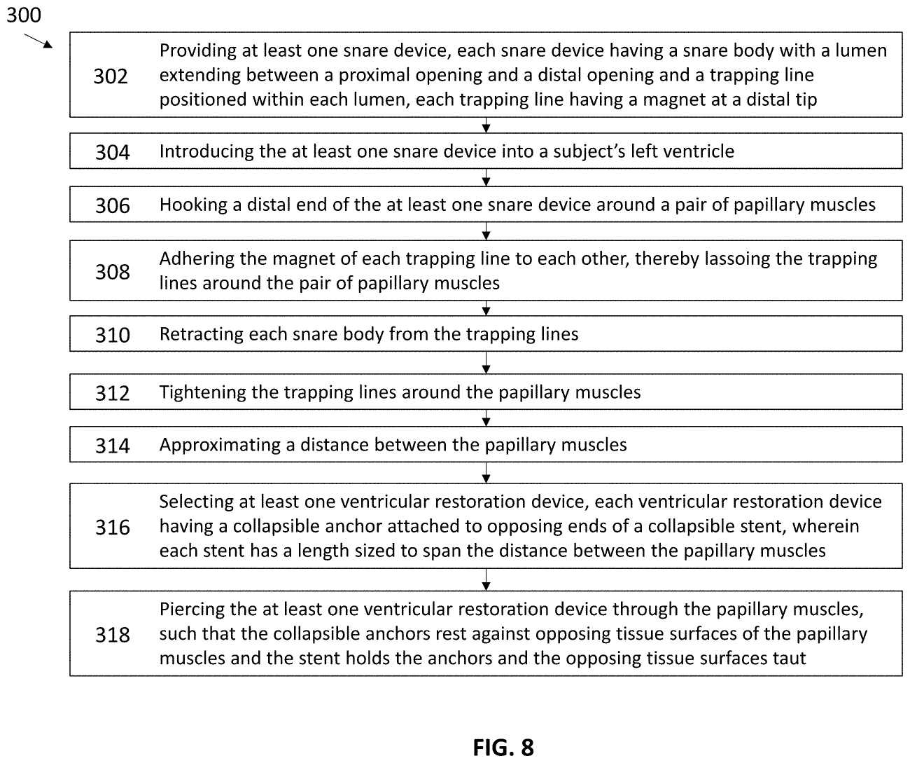

The present invention further includes methods of repairing mitral regurgitation (MR) using the ventricular restoration devices and snare devices of the present invention. The method can be performed without open heart surgery, as the ventricular restoration devices and snare devices are compatible with minimally invasive hardware and procedures. Referring now to FIG. 8, an exemplary method 300 is depicted. Method 300 begins with step 302, wherein at least one snare device is provided, each snare device having a snare body with a lumen extending between a proximal opening and a distal opening and a trapping line positioned within each lumen, each trapping line having a magnet at a distal tip. In step 304, the at least one snare device is introduced into a subject's ventricle. In step 306, a distal end of the at least one snare device is hooked around a pair of papillary muscles. In step 308, the magnet of each trapping line is adhered to each other, thereby lassoing the trapping lines around the pair of papillary muscles. In step 310, each snare body is retracted from the trapping lines. In step 312, the trapping lines are tightened around the papillary muscles. In step 314, a distance between the papillary muscles is approximated. In step 316, at least one ventricular restoration device is selected, each ventricular restoration device having a collapsible anchor attached to opposing ends of a collapsible stent, wherein each stent has a length sized to span the distance between the papillary muscles. In step 318, the at least one ventricular restoration device is pierced through the papillary muscles, such that the collapsible anchors rest against opposing tissue surfaces of the papillary muscles and the stent holds the anchors and the opposing tissue surfaces taut.

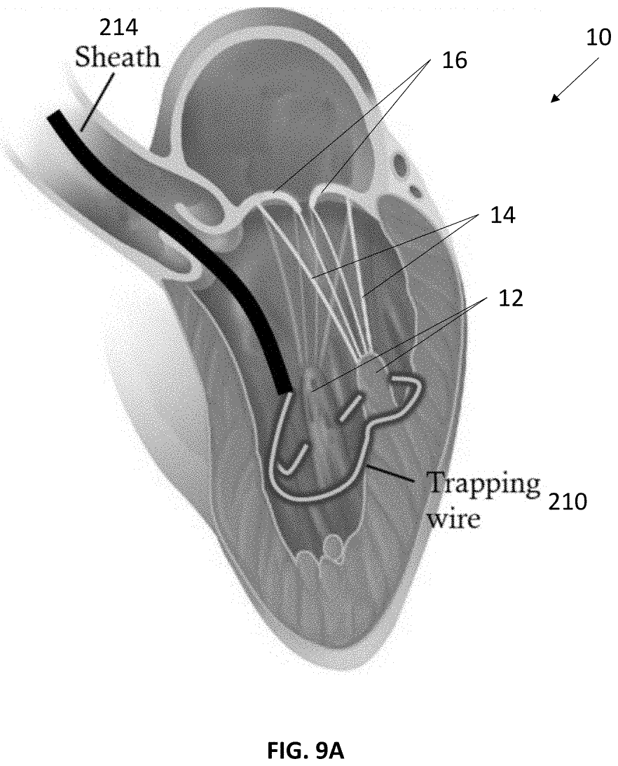

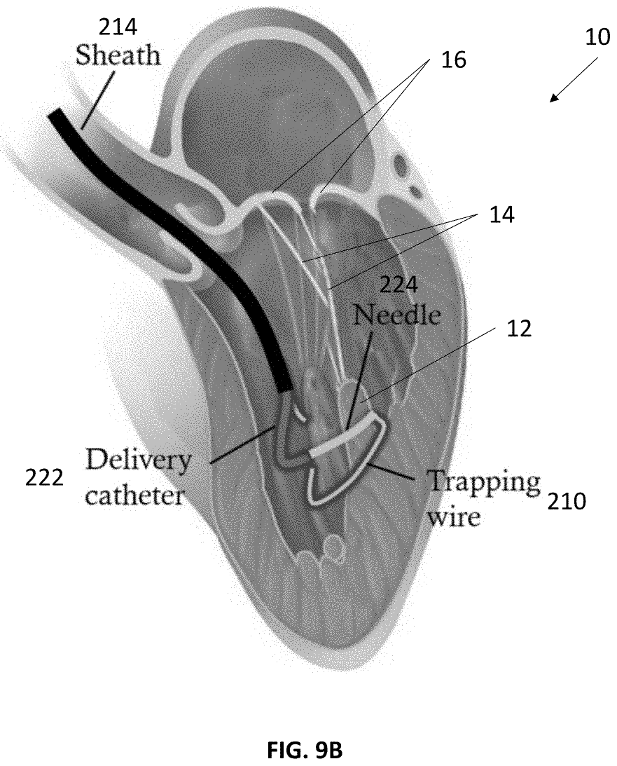

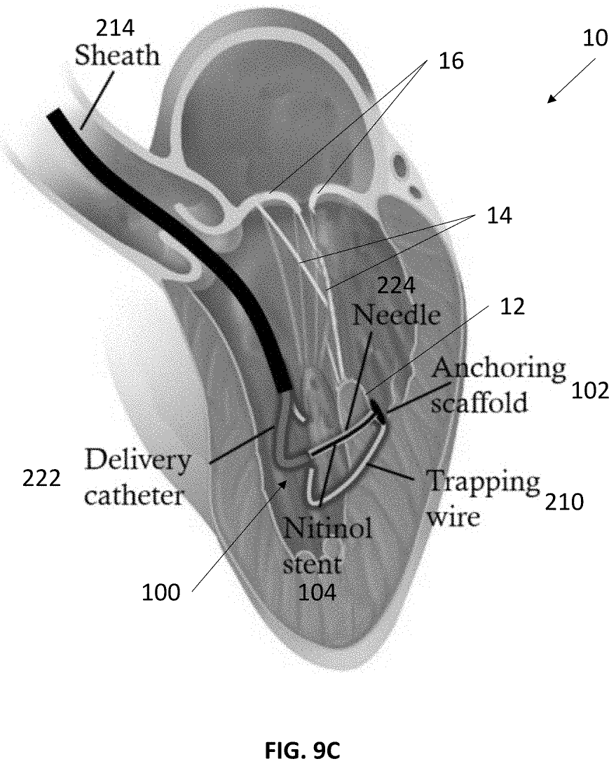

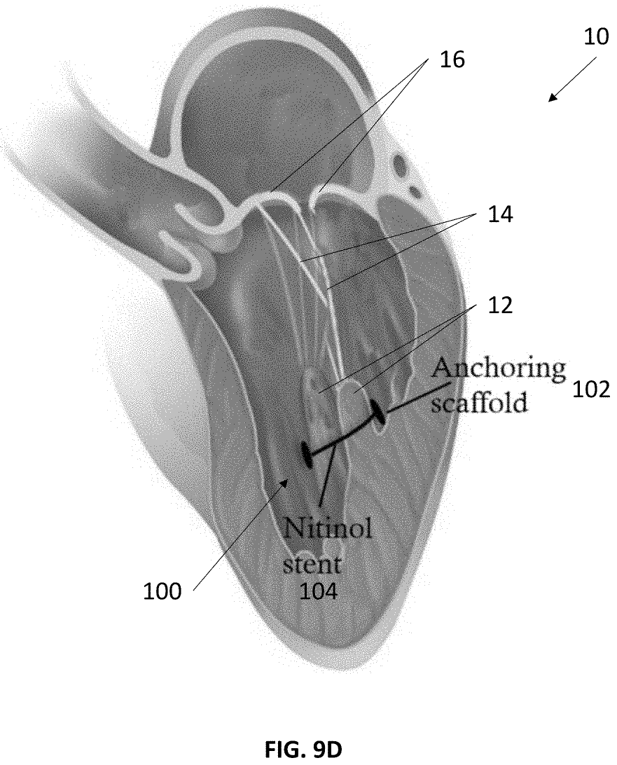

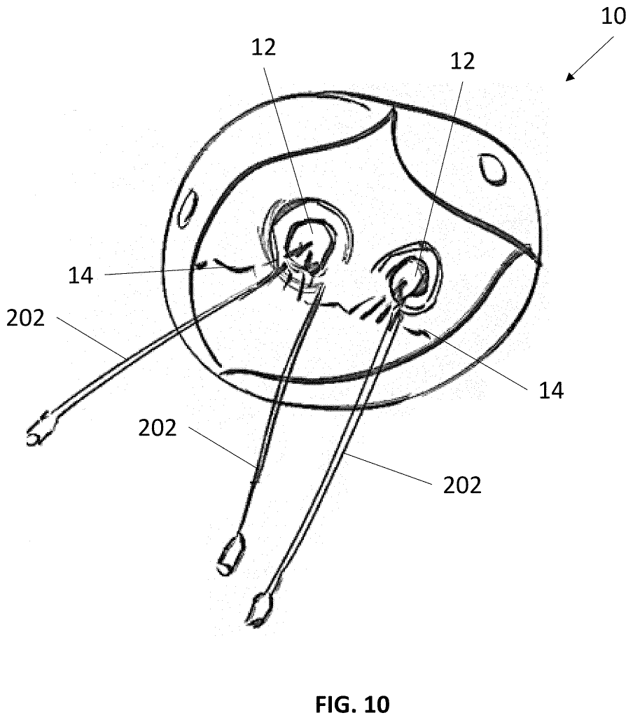

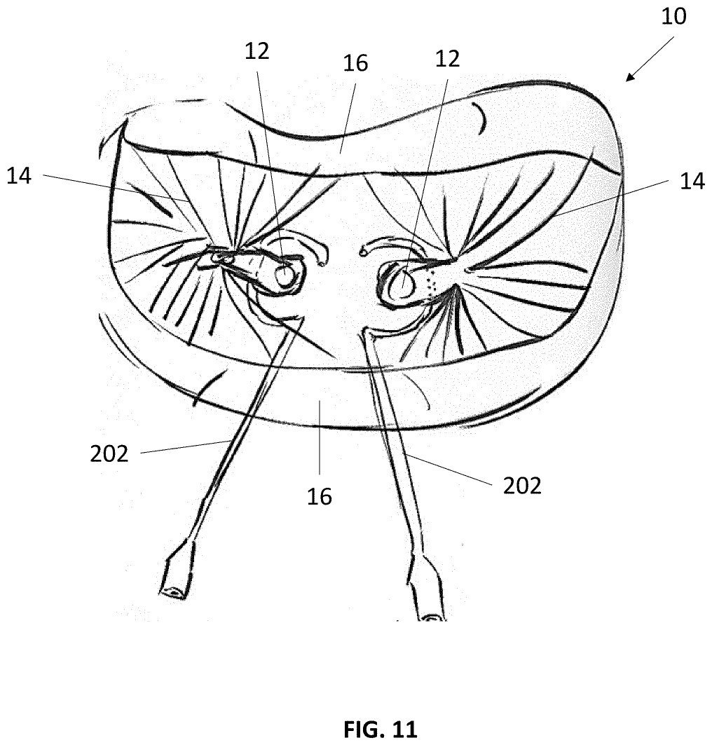

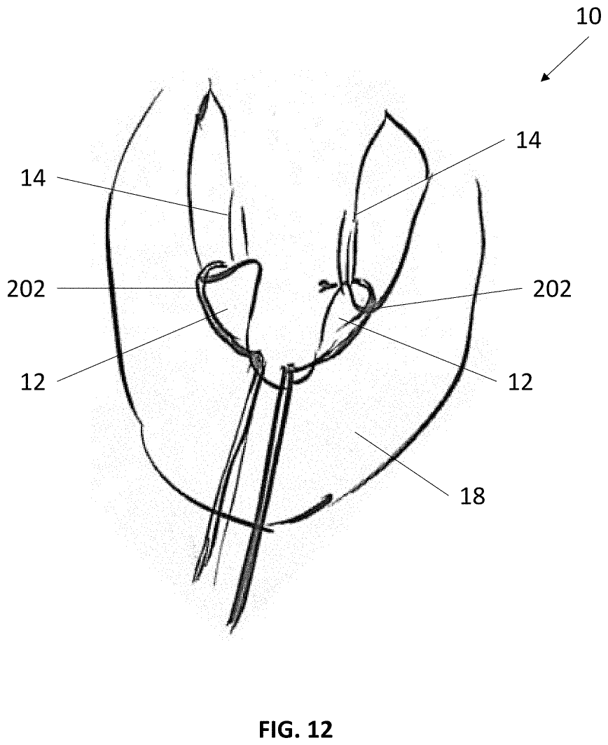

FIG. 9A through FIG. 9D depict the steps of method 300. FIG. 9A depicts trapping lines 210 lassoed around the papillary muscles 12 after snare bodies 202 have been retracted into catheter 214. FIG. 9B depicts trapping lines 210 tightened around the papillary muscles 12. MR can be evaluated to determine whether the tightened position of the papillary muscles 12 is effective. If it is not effective, trapping lines 210 can be loosened, repositioned, and tightened to determine a more effective position. Once an effective position has been determined, a needle 224 can be introduced via a delivery catheter 222 to pierce the papillary muscles 12. FIG. 9C depicts device 100 threaded over needle 224 to pierce the papillary muscles 12. A distal anchor 102 is shown having expanded from a collapsed state upon exiting a distal tissue surface of a papillary muscle 12. Once device 100 is in place, delivery catheter 222 can be removed to expand an opposing proximal anchor 102. FIG. 9D depicts device 100 in place holding the papillary muscles 12 in place with all other tools and devices removed. The ventricle can be accessed using any suitable method. For example, FIG. 10 depicts a transaortic approach, FIG. 11 depicts a transmitral approach, and FIG. 12 depicts a transventricular approach.

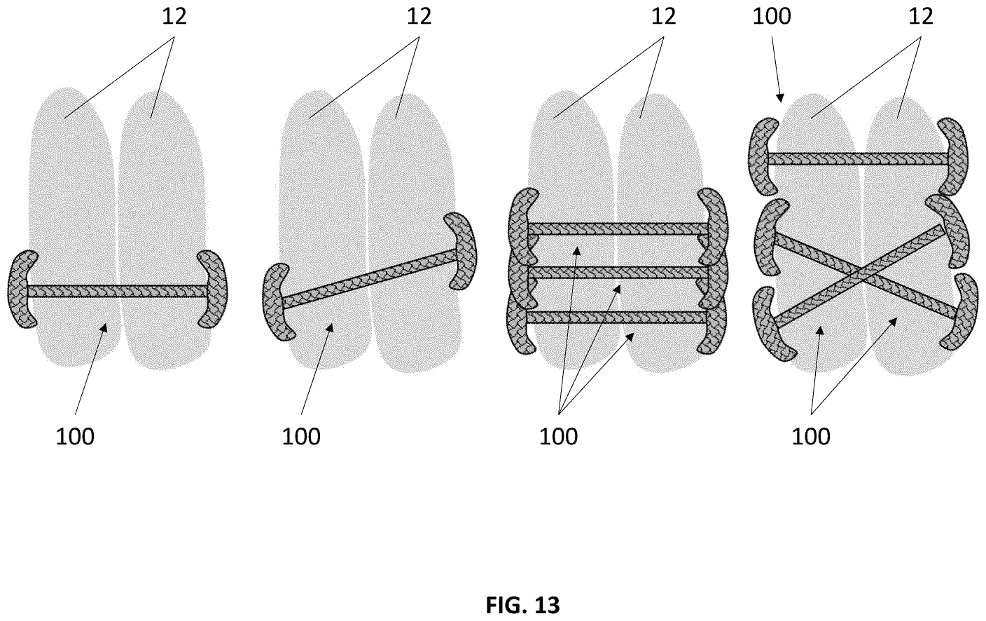

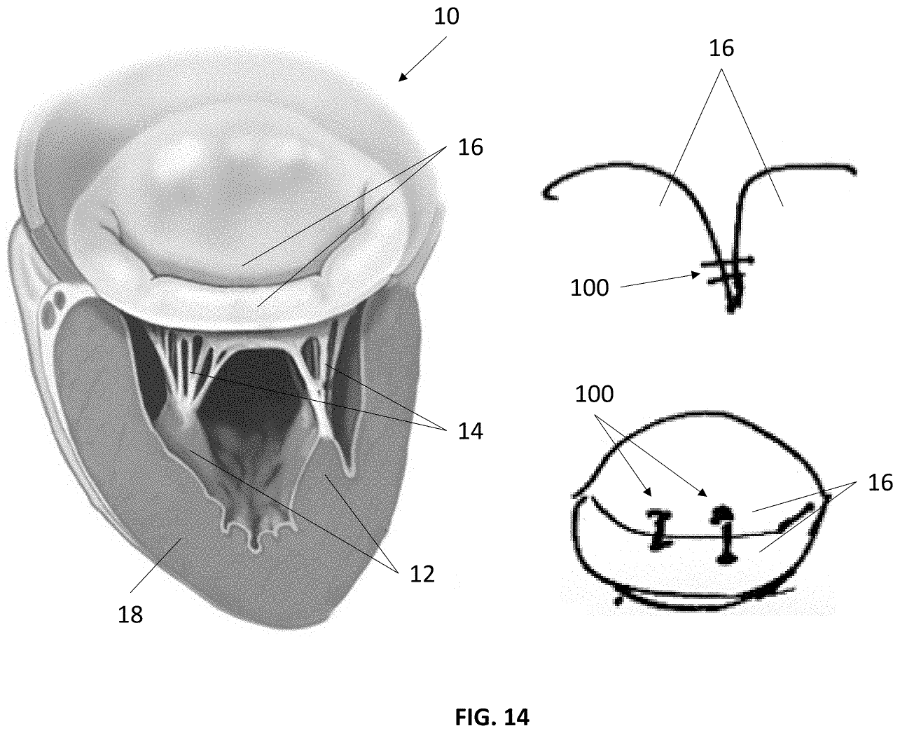

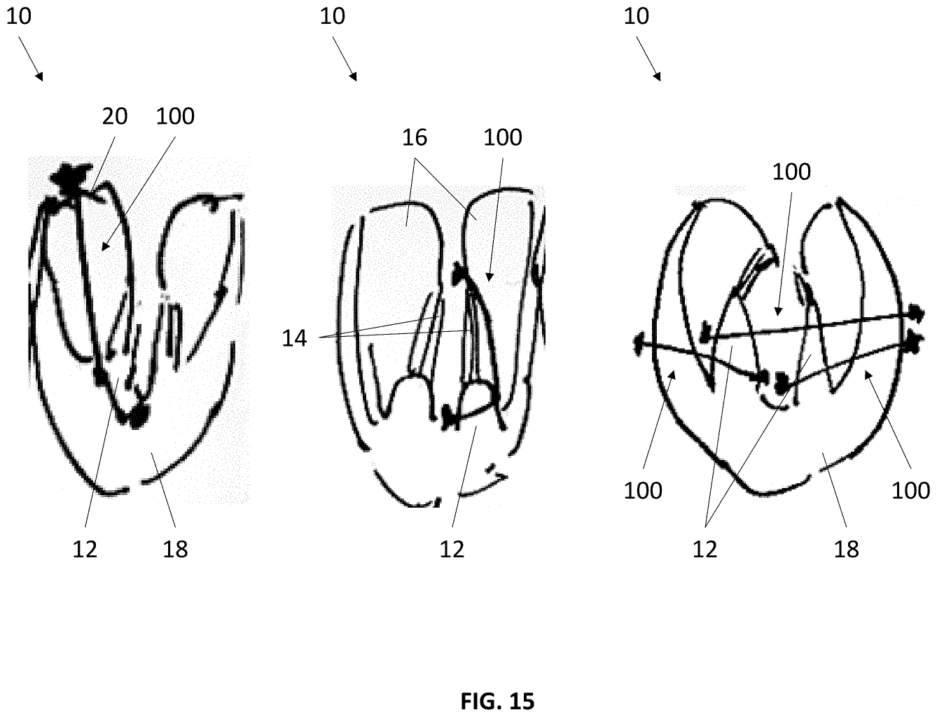

As described elsewhere herein, the ventricular restoration devices of the present invention are not limited in implant orientation or combination. For example, FIG. 13 depicts one or more devices 100 implanted transversely, at an angle, or combinations thereof between adjacent papillary muscles 12. It should also be understood that the ventricular restoration devices of the present invention are not limited to implanting in papillary muscles. For example, FIG. 14 depicts one or more devices 100 implanted in leaflets 16. Devices 100 can be used to secure leaflets 16 from the side (top right) or across the leaflets 16 (bottom right). FIG. 15 depicts one or more devices 100 implanted in various configurations in a subject's heart. For example, devices 100 can be used to secure a portion of a valve annulus 20 to a papillary muscle 12 (left). Devices 100 can also be used to augment the chordae tendinae 14 by securing a papillary muscle 12 to a valve leaflet 16 (middle). Devices 100 can also be used to alter the shape of the ventricle by securing papillary muscles 12 and cardiac muscle 18 in various configurations (right). In embodiments comprising devices 100 having an array of magnetic anchors 102, one or more additional magnetic anchors 102 may be secured or implanted in various locations in a subject's heart to bias the compressed papillary muscles 12 in any desired direction.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A papillary muscle approximation and ventricular restoration (PAP-VR) system comprising:
at least one ventricular restoration device, each ventricular restoration device comprising a collapsible anchor attached to opposing ends of a collapsible stent; and
at least one snare device, each snare device comprising an elongate snare body having a lumen connecting a proximal opening and a distal opening, and a trapping line positioned within the lumen, each trapping line having a magnet attached to a distal end.

2. The system of claim 1, wherein the at least one ventricular restoration device further comprises one or more magnetic anchors, wherein the one or more magnetic anchors are configured to form a band or ring like shape upon magnetic attraction to each other.

3. The system of claim 2, wherein the one or more magnetic anchors each comprise a papillary muscle-facing surface comprising a plurality of barbs or hooks.

4. The system of claim 2, where the one or more magnetic anchors are linked together by a fabric, a mesh, or a sleeve.

5. The system of claim 1, wherein each of the anchors has a shape selected from the group consisting of: a donut shape, a bracket shape, a claw shape, a barb shape, an orthogonal rod shape, a cross shape, a multi-legged shape, a disc shape, a clover shape, and combinations thereof.

6. The system of claim 1, wherein the at least one ventricular restoration device is constructed from a material selected from the group consisting of: nitinol, PTFE, polyester, and silicone.

7. The system of claim 1, wherein a distance between each opposing anchor is adjustable along a length of each stent.

8. The system of claim 1, wherein each snare device comprises a distal hook end.

9. The system of claim 1, wherein each snare device comprises a main axis aligned along a length of each snare body.

10. The system of claim 8, wherein each snare body comprises at least one out-of-axis or out-of-plane angulation that deviates from the main axis.

11. The system of claim 9, wherein the at least one out-of-axis or out-of-plane angulation is positioned along the length of the snare body, at a junction between a distal hook end, along a curvature of the distal hook end, and combinations thereof.

12. A method of repairing mitral regurgitation in a subject, the method comprising the steps of:
providing at least one snare device, each snare device having a snare body with a lumen extending between a proximal opening and a distal opening and a trapping line positioned within each lumen, each trapping line having a magnet at a distal tip;
introducing the at least one snare device into a subject's left ventricle;
hooking a distal end of the at least one snare device around a pair of papillary muscles;
adhering the magnet of each trapping line to each other, thereby lassoing the trapping lines around the pair of papillary muscles;
retracting each snare body from the trapping lines;
tightening the trapping lines around the papillary muscles;
approximating a distance between the papillary muscles;
selecting at least one ventricular restoration device, each ventricular restoration device having a collapsible anchor attached to opposing ends of a collapsible stent, wherein each stent has a length sized to span the distance between the papillary muscles; and
piercing the at least one ventricular restoration device through the papillary muscles, such that the collapsible anchors rest against opposing tissue surfaces of the papillary muscles and the stent holds the anchors and the opposing tissue surfaces taut.

13. The method of claim 12, wherein the left ventricle is accessed from a transaortic approach, a transmitral approach, or a transventricular approach.

14. The method of claim 12, wherein the approximating step comprises a step of calculating a total length of trapping line lassoed around the pair of papillary muscles such that the total length forms a circumference of a substantially circular shape.

15. The method of claim 14, wherein the selection step selects at least one ventricular restoration device having a length substantially equal to a diameter of the substantially circular shape.

16. The method of claim 12, further comprising a step of piercing at least one ventricular restoration device through a papillary muscle and a valve annulus.

17. The method of claim 12, further comprising a step of piercing at least one ventricular restoration device through a papillary muscle and a valve leaflet.

18. The method of claim 12, further comprising a step of piercing at least one ventricular restoration device through a papillary muscle and a heart wall, such that the papillary muscle is shifted out of its natural plane.

19. The method of claim 12, wherein the at least one ventricular restoration device comprises one or more magnetic anchors.

20. The method of claim 19, further comprising a step of implanting at least one magnetic anchor into an adjacent heart wall, such that magnetic attraction between the at least one ventricular restoration device and the at least one magnetic anchor in the heart wall biases the position of the at least one ventricular restoration device.
